(19) United States
(12) Patent Application Publication
(10) Pub. No.: US 2013/0089465 A1
(43) Pub. Date: Apr. 11, 2013

(54) MICROMECHANICAL SENSOR SYSTEM HAVING SUPER HYDROPHOBIC SURFACES

(71) Applicants: Marco LAZZARINO, Trieste (IT); Mauro MELLI, Trieste (IT)

(72) Inventors: Marco LAZZARINO, Trieste (IT); Mauro MELLI, Trieste (IT)

(73) Assignee: CNR CONSIGLIO NAZIONALE DELLE RICERCHE, Rome (IT)

(21) Appl. No.: 13/270,975

(22) Filed: Oct. 11, 2011

(51) Int. Cl. G01N 33/53; G01N 21/63

(52) U.S. Cl. 422/69

(57) ABSTRACT

A sensor system is provided to detect the mass of a compound in a liquid solution, the system including a sensor including a plurality of pillars extending from a substrate and having a given height, the pillars having a free end opposite to the substrate, and including a lateral surface connecting said free end to the substrate. The free end defining a surface and the surface is functionalized in order to bind with the compound to be detected, and the lateral surface is hydrophobic. The distance between any two nearest neighbors pillars of the plurality satisfies the following equation

$$\frac{height of any of the two n . n . pillars}{maximum distance between the two n . n . pillars} > 1.$$

The system also includes a detection device to detect the oscillations of said pillars.

### FIELD OF THE INVENTION

The present invention relates to a mechanical sensor system, in detail a Microelectromechanical system (MEMS), to detect the concentration of compounds or other particles in liquids, and more in particular aqueous solutions. The sensor system includes a plurality of resonators arranged in an ensemble which has super-hydrophobic properties.

### BACKGROUND ART

MEMS is the integration of mechanical elements and electronics on a common substrate through the utilization of microfabrication technology. The mechanical part, which can move, has two main functions sensing and actuating. For example, MEMS are used as accelerometers, gyroscopes, and pressure or flow sensors and, as actuator, they are use as micromotors, mirror mounts or micro pumps.

The term BioMEMS was introduced to specify a class of MEMS used for biological application but nowadays it has a more broad and general meaning. R. Bashir in his review about BioM EMS gives the definition “devices or systems, constructed using techniques inspired from micro/nanoscale fabrication, that are used for processing, delivery, manipulation, analysis, or construction of biological and chemical entities”. According to this classification, for example, also force microscopy based on atomic force microscopy or microfluidics devices can be categorized as “BioMEMS”.

The Atomic Force Microscope (AFM) was invented in 1986 as tool for imagining surfaces. It is a complex instrument but the sensing element is simple and completely mechanical. In fact, it is just a cantilever which bends because of interactions with the substrate. In the last few years non-imaging applications were developed and the AFM was used for studying the inter- and intra-molecular interactions down to the single molecule level. This became possible because force spectroscopy based on these new experimental tools allows measuring force in the piconewton range on the ms time scale. The AFM force spectroscopy (i.e. a cantilever with a tip) is still a growing field but researchers have also begun to develop cantilever-only mechanical sensors. A cantilever is only one of the possible geometries of a mechanical molecular sensor, an oscillating bridge being another.

These Bio-MEMS are extremely interesting because they allow to detect proteins, such as analytes in a fluid. This is possible due to the high accuracy achieved in detecting the presence of a mass (such a molecule) in solutions. In addition, those sensors can detect the type of molecule present in the fluid, again due to the possible differentiation of molecules depending on their masses.

A review of the techniques and bio-sensors used nowadays is given in the article published in Nature Nanotechnology the 11^{th} of March 2011, entitled “Comparative advantages of mechanical biosensors” by J. L. Arlett, E. B. Myers and M. L. Roukes. A list of different techniques is given with their advantages and limitations. In particular, an outstanding challenge in biosensing is to engineer suites of reliable, high-affinity biochemical agents to capture the target biomarkers we are interested in detecting. High affinity binding is based on biological molecular recognition, which generally occurs only in liquid phase. After capture, target detection is ideally performed in situ, within the fluid. However alternative approaches include removing the detector from the fluid (after the targets are captured), and desiccating it before measurement. Detection in situ is obviously simpler and immediate, but mechanical sensing in fluid is strongly affected by viscous damping and this significantly reduces the mass resolution compared with that obtained in gas or vacuum.

The US patent application US 2010/0107285 describes tunable, bio-functionalized, nanoelectromechanical systems (Bio-NEMS), micromechanical resonators (MRs), nanomechanical resonators (NRs), surface acoustic wave resonators, and bulk acoustic wave resonators having super-hydrophobic surfaces for use in aqueous biochemical solutions. The MRs, NRs or Bio-NEMS include a system resonator that can vibrate or oscillate at a relatively high frequency and to which an analyte molecule(s) contained in the solution—can attach or upon which small molecular-scale forces can act; a device for adjusting a relaxation time of the solution, to increase the quality (Q-factor) of the resonator inside the solution, to reduce energy dissipation into the solution; and a device for detecting a frequency shift in the resonator due to the analyte molecule(s) or applied molecular-scale forces. The resonator can include roughness elements that provide super-hydrophobicity and, more particularly, gaps between adjacent asperities for repelling the aqueous solution from the surface of the device.

### SUMMARY OF THE INVENTION

The present invention relates to a sensor system for the detection of particles, in particular proteins, even more particularly bio-markers, for example from samples of biologic fluids.

A typical area of interest is the detection of cancer markers.

The principle on which the sensor system is based is discussed in the following. A mechanical physical system—such a cantilever or a pillar—responds to an external oscillating force with different amplitudes as a function of the frequency. The spectral distribution is characterized by peaks which are known as resonant frequencies which correspond to oscillating modes. A small driving force at resonance can induce a large oscillation. It is shown that a load at the end of a cantilever induces a deflection which is linearly proportional to the force applied. Therefore, it is natural to introduce a lumped element model to describe the dynamics of a cantilever. The cantilever is approximated a mass linked to a spring (characterized by the spring constant k) which moves in a viscous medium. In order to describe all the geometrical effect due to a tridimensional structure, the actual values of the mass is substituted by a reduced mass value. Each mode has different geometrical factors. A resonance curve is characterized by two parameters: the position of the peak (resonance frequency) and the width of the peak which is generally calculated at the half maximum and indicated with FWHM (Full Width at Half Maximum). More often, a dimensionless parameter, the quality factor or Q factor is used. It is defined as the ratio between the resonance frequency and the FWHM. Typical ranges are in the prior art for example Q=10000 in vacuum, Q=10-500 in air, and Q<5 in water. The Q factor has also a physical interpretation, being proportional to the ratio between the energy stored to the energy being lost in one cycle. If the damping is negligible, the peak position f of the lowest mode corresponds to the natural frequency and is given by:

$${f}_{D} = \frac{1}{2 π} \sqrt{\frac{k}{m}} = \frac{1}{2 π} \sqrt{\frac{E}{ρ}} \frac{t}{L} \qquad ( 1 )$$

where

K=elastic constant,

m*=reduced (or effective) mass,

E=young modulus,

ρ=density of the material which compose the cantilever,

t=thickness of the cantilever, and

L=length of the cantilever.

The adsorption of molecules changes the shape of the resonance curves. The main effect is to shift the resonance frequency. As first approximation, the mass of the resonator increases by the quantity Δm which corresponds to the mass of adsorbed molecules. According to the harmonic oscillator equation, the resonance changes to the value:

$${f}_{D} = \frac{1}{2 π} \sqrt{\frac{k}{{m}^{-} + {}_{Δ} m}} \qquad ( 2 )$$

However this is an approximation that does not take into account two very important physical processes: the variation depends on the position where the adsorption takes place and the adsorbed molecules affect the elastic properties of the beam. A more accurate equation is:

$${f}_{D} = \frac{1}{2 π} \sqrt{\frac{k [ Δ m ]}{{m}^{-} + {γ}_{Δ} {m}^{0}}} \qquad ( 3 )$$

where now k is a function of the adsorbed mass and γ is a geometrical parameter determined by the location of absorption. So, it is clear that measuring only the resonance frequency does not provide any quantitative result.

Alternatively, it is possible to focus on the change of other parameters like the change of the device compliance or the Q factor. This technique is extremely sensitive in vacuum (attogram resolution) but has the big disadvantage that in a liquid environment loses its power. Due to viscous effect the width the resonance increases and the amplitude decreases dramatically, as seen above (Q<5). The main consequence is that minimum detectable frequency shift becomes very large and this approach becomes useless. For biological application, this is a huge limitation but one possible solution is to separate the functionalization and adsorption phase from the measuring phase. This approach is commonly known as “dip and dry”. All the chemical reactions are performed in solution then the device is dried and placed in a vacuum chamber where the resonance frequency is measured. With this procedure, it is possible to preserve high sensitivity but becomes necessary to renounce to real time detection.

The goal of the invention on the other hand is to keep the high accuracy, but to also obtain real time detection, without using the “dip and dry” technique. Indeed, the present invention overcomes these problems, allowing a real time detection in a liquid environment and at the same time having a high Q factor (i.e. substantially the same Q factor as in a gas).

Preferably the sensor system of the invention includes a sensor, a detection device of the sensor movements and optionally an actuator.

The sensor includes a plurality of substantially vertical pillars protruding from a substrate, which is preferably planar. Preferably, the substrate is a silicon wafer or a crystalline silicon. However many materials are suitable in addition to the preferred ones. The most used other than silicon are silicon carbide, silicon nitride, carbon compound (including polymers), III-V compounds and all the materials which are easily fabricated with standard lithography and etching processes and offer a high young modulus. Another common substrate in MEMS technology is silicon on insulator (SOI). It consists in a thick wafer of silicon covered by a thin thermal silicon oxide and of a thin crystalline layer of silicon which has the same crystallographic orientation of the substrate.

In order to obtain the pillars, the three fundamental processes, lithography, etching and film deposition, are preferably used. Therefore, preferably substrate and pillars are realized in the same material, and the list has been given above.

The pillars have a given height H, which is preferably comprised between 5 μm and 50 μm. Pillars are so realized that their vertical extension, i.e. their height calculated from the substrate to which they are attached, is much bigger than their other two dimensions of the cross section. The height of the pillars in the plurality can be substantially the same among all pillars, however also pillars having different heights can be used in the present invention as long as the equations below explained are satisfied. The geometrical distribution of the pillars can be ordered or disordered (i.e. random). For example pillars can form an hexagonal or quadratic configuration, or they can be arranged in a substantially random or quasi-random distribution. However in any formed pattern, the maximum distance between any two nearest neighbor pillars of the plurality is shorter than the height of any of the two nearest neighbor pillars considered. Preferably, the distance between any two nearest neighbor pillars is comprised between 2 μm and 50 μm, more preferably between 5 μm and 40 μm. The definition of the distance between two nearest neighbor pillars is the following: the distance between the geometrical centers of the two pillars is calculated.

The distance between any couple of nearest neighbor pillars in the plurality can be always the same or it can vary within a certain range. In a ordered lattice for example, said distance can be fixed, while in a random lattice can be random as long as the above mentioned characteristic is satisfied.

The pillars act as the resonators (the functioning of which has been above described with reference to the prior art) and their change in frequency of the resonance is checked to detect and identify the type of molecules, or more in general compounds (particles or aggregates), which come into contact with the sensor, as better detailed below.

Preferably the pillars have a rectangular cross section, however any cross section can be considered as well for the application of the present invention. Preferably, the width of the pillars at their base, i.e. where the pillar is attached to the substrate, is comprised between 30% and 100% of the width at the opposite free end. With the word “width” the smallest internal dimension of the pillar in cross section is meant.

Pillars define a free end opposite to the end attached to the substrate. Said free end includes a free surface substantially parallel to the substrate and located at the height H (the height of the pillar) from the latter, and it has an area preferably comprised between 0.2 μm² and 50 μm². In addition, each pillar includes a lateral surface, which may comprise a plurality of facets if the pillars have the shape of a parallelepiped or it may comprise a cylindrical envelope in case of cylindrical pillar, however other geometries are envisaged as well. In other words, the lateral surface is the surface connecting the free surface to the substrate.

Said lateral surface can be perpendicular to the end surface and to the substrate, however tilted pillar or frusto-conical pillars can be envisaged as well. Preferably, the pillar is frusto-conical, having a cross sectional area which increases starting from the substrate (the base has the smallest area) towards the free end surface (which has the widest area). Preferably the angle formed by the lateral surface and the substrate is comprised between 3° and 6°. The surface finishing of said lateral surfaces can be either flat or rough, with roughness deriving from the etching process used to fabricate the pillar. Roughness can be characterized by nanoscale porosity and superstructures. Root Mean Square (RMS) roughness comprised between 1 nm and 10 nm is preferred to assist the formation of a superhydrophobic surface as described below.

The free surface of each pillar is functionalized. With the term “functionalization”, in the present context the following is meant: the free top surface of the pillar is treated chemically, preferably a layer of molecules is formed, and more preferably a layer of oriented biomolecules such antibodies, in order to make the functionalized surface able to react selectively and capture a specific compound, or bind, such as a molecule or analyte, which is the target to be detected or measured or identified.

In a first example, on top of the free end surface of each pillar a metal layer is deposited, for example by a directional deposition system, such as thermal evaporation.

As an example, with this coating, there is an automatic self-alignment at the very end of the resonator without the use of any lithographic tool. The fabrication process can thus be pushed to its intrinsic limit without loss of alignment precision. By choosing a specific interaction. i.e. selecting the type of analyte to be detected, typically gold-thiol, the adsorption is localized on the metal surface which corresponds exactly to the top free end surface of the pillar. The adsorbed analyte does not induce any stress on the oscillating part of the pillar that corresponds to the lateral surface, in case of a parallelepiped pillar the side walls. Moreover, all mass is localized exactly at the end of the beam and the spring model can be correctly applied in order to associate the change in frequency with adsorption.

Furthermore, the fabrication process is intrinsically symmetrical, so that all the vertical walls are equally finished and no asymmetrical residual stresses are induced by fabrication as in the case of horizontal geometry.

In a second example, the silicon surface of the pillar is treated with siloxane molecules that carry at the other end (the end not linked to the free end surface) a functional group that in turn can be linked to the antibody of interest. The surface so treated will thus expose a protein layer—better an antibody layer—that recognize specifically the antigens to which the chosen antibody offer a high binding affinity.

In a third example, a layer of gold is deposited on the top surface of the pillars, exploiting the directionality and selectivity of the pillar design. The Au layer is immediately passivated (which means the available sites for chemical bonds are saturated i.e. occupied by new molecular bonds) with thiolated (which means sulphur terminated) biomolecus, preferentially thiolated antibodies.

In addition to the functionalization, the free surface of the pillars is hydrophilic. The functionalization of said surface can be identical for all pillars. Alternatively and preferably every pillar can be functionalized to recognize a different protein or specifically a different biomarker in a matricial configuration. More specifically, each pillar can be indexed to localize the signal and associate it to a specific biomarker. A compact fingerprint assay can be thus implemented. As a third options, groups of pillars can have the same functionalization and different groups have distinct functionalization to both improve statistical signal to noise ratio and detect a large number of different biomarkers in a finger assay configuration.

In addition, the lateral surface of the pillars, i.e. the side walls, are treated in such a way to make it hydrophobic. Any treatment is possible, as long as the resulting surface is hydrophobic while the free functionalized end is hydrophilic. Possible treatments are coating the lateral surface with a water-repellent material, such as Teflon (Polytetrafluoroethylene (PTFE)), or a coating non-polar terminated chlorosilanes, the latter being the preferred method of the invention. The abovementioned surface roughness can be used to increase the hydophobicity of said lateral surface by increasing the actual surface area and thus increasing the energy required to wet said lateral surface.

The combination between the specific geometry of the system, as better detailed below, and the lateral surface's treatment above described renders the whole sensor system superhydrophobic. A superhydrophobic surface is that surface which is extremely difficult to wet. The contact angles of a water droplet exceeds 150° and the roll-off angle is less than 10°. This is referred to as the Lotus effect. In the present invention this effect is obtained arranging the plurality of pillars in the plurality in such a way that the distance between any couple of pillars which are nearest neighbors in the plurality satisfies the following equation:

$$\frac{height of any of the two pillars}{maximum distance between the two pillars} > 1 \qquad ( 4 )$$

Preferably,

$$\frac{height of any of the two pillars}{maximum distance between the two pillars} < 5. \qquad ( 5 )$$

These equations (4) and (5) are valid for any couple of nearest neighboring pillars of the plurality, therefore selected a single pillar, all its nearest neighbors are located at a distance lower than the height of the selected pillar.

Additionally, the plurality of pillars is preferably surrounded by a wall. Said wall encloses all pillars and defines an inner surface of the substrate where all the pillars are present and an “outside” surface of the substrate external to the sensor. Preferably, the height of the wall is substantially identical to the height of the pillars, or in case the pillars' heights are within a given range, the wall height is within the same range. Additionally, preferably the thickness of the wall is larger than 1 μm. Eq. (4) or eq. (5) also applies in relation of the maximum distance between the pillars of the plurality and the wall: the relationship between the wall and any of its nearest neighbors (n.n.) pillars to the wall is the following:

$$\frac{height of the nearest neighbor pillar and the wall}{maximum distance between the wall and n . n . pillars} > 1 \qquad ( 6 )$$

more preferably

$$2 < \frac{height of the nearest neighbor pillar and the wall}{maximum distance between the wall and n . n . pillars} < 5 \qquad ( 7 )$$

When the plurality of pillars is put into contact with a fluid, wherein the substance to be the detected is present, the following phenomenon takes place. Due to the achieved super hydrophobicity as explained above, the substrate and the lateral surface of the pillars are not wetted by the fluid, on the contrary they remain in contact with air or a suitable gas mixture to decrease the viscous damping, preferably a non interacting Gas such as Helium or Argon, depending on the system used. Alternatively, the sensor system can be kept in vacuum and pillars can resonate as in vacuum. Only the functionalized top free surfaces of the pillars get in contact with the liquid. The extension of the area of each top free end compared to the extension of the overall area of the pillar is rather limited (the overall area includes the top free surface and the lateral surface, the latter being in general much larger than the former) and—due to this—each pillar of the sensor system is oscillating substantially as in air, i.e. the amount of contact between the liquid which is injected into the system and the pillar does not substantially change the Q factor of each pillar. In this way, a real time detection can be made which is extremely accurate: the measurement is made while the pillars are in contact with the liquid and at the same time the same accuracy obtained in dry conditions can be achieved.

Therefore the simplified equation (2) can be used and the resulting Q factor is substantially analog to the Q factor in air. The dampening effect of the fluid is not seen, indeed the fluid is wetting only a very small fraction of the pillar.

The surrounding wall which encircles the pillars in addition prevents the pillars' lateral surfaces and the substrate from getting wetted, confining the pillars in an enclosed area and preventing lateral injection of fluid.

In order to obtain the mass measurements desired of the target compound(s), such as molecules or elements present in the fluid, the sensor system includes at least a pillar which acts as a resonator, the pillars are put into contact with a fluid where the compound to be measured is present and the change in resonance frequency of at least one pillar is checked. This check is performed using a detecting device.

To detect the frequency response of the pillars in real time upon molecules adsorption, the sensors are included in a microfluidic device described in FIG. 1. Here the chip containing the pillars fabricated and functionalized are the base of a microfluidic chamber, the wall defines laterally the microfluidic chamber, an inlet and an outlet port are located at opposite sides of the chip and are connected to a pumping system suitable for make the liquid circulating in the device at a suitable flow. The microfluidic chamber volume is comprised preferably between 0.01 nL to 10 nL, more preferably between 0.1 nL to 1 nL. The top wall of the microfluidic chamber is realized at least partially by an optical window which is transparent to the wavelength used in the detection procedure. The optical window finishing is preferably of optical quality with the internal side preferentially coated with hydrophobic molecules to decrease non specific adsorption of target compounds. The window thickness is preferably comprised between 0.01 mm to 10 mm, preferentially between 0.1 mm and 1 mm.

The detection device of the present invention can be of any type. They can be optical or electrical. Preferably, the optical lever method is used. The working principle is based on deflection of a laser spot deflected at the focus on the free surface of the pillar(s) of the sensor. The angular deflection of the laser beam is twice that of the pillar. The reflected laser beam strikes a position-sensitive photodetector consisting of two side-by-side photodiodes. The difference between the two photodiode signals indicates the position of the laser spot on the detector and thus the angular deflection of the cantilever. Because the pillar-to-detector distance generally measures thousands of times the length of the pillar, the optical lever greatly magnifies (˜2000-fold) the motion of the tip. Photodiodes divided into two or four independent areas transduce the light into electrical signals that are amplified and elaborated to get the deflection of the beam. To detect a number of pillars in parallel alternative techniques can be employed such as sample scanning, laser scanning, multi beam lasers. Any other detection method is however possible

An actuator is optionally used in the present sensor to obtain the highest possible sensitivity, in particular to increase the signal to noise ratio. The sensor system includes a chip on which the sensor is mounted which is in turn fixed directly onto a piezoelectric crystal. In other words, the pillars are put into oscillation. In this method there is not any direct force acting on the pillars but rather a mechanical coupling between the movement of the piezo and the modes of the pillars. This technique is able to actuate at frequencies lower than few MHz due to intrinsic frequency cut off the piezo.

Also in the US application 2010/0107285 a sensor in liquid environment is described, however such a sensor still resonates in liquid and its Q factor is indeed rather low. The pillars in this sensor are considered as “roughness” and not sensors themselves. The real resonator is the cantilever including the pillars, not the pillars. This still results in an oscillation in liquid and thus a big dampening effect and low sensitivity.

### BRIEF DESCRIPTION OF THE DRAWINGS

The invention will be better described and understood with reference of the appended drawings in which:

FIG. 1 is a schematic lateral view of a sensor system realized according to the present invention;

FIG. 2 is a SEM photograph of a single pillar part of the sensor system of FIG. 1 of the present invention;

FIG. 3 is a SEM photograph of the sensor included in the sensor system of FIG. 1;

FIG. 4 is a schematic representation of a liquid drop on top of the pillars of the sensor system of FIG. 1;

FIG. 5 are two graphs reporting the normalized amplitude versus the frequency shift for a pillar with (above curve) and without (below curve) being in contact with a liquid drop;

FIG. 6 is a schematic view of the different steps to obtain a portion of the sensor system of the invention;

FIG. 7 is a schematic representation of the detecting apparatus using the sensor system of the present invention;

FIG. 8 is an additional SEM photograph of the pillar of the present invention;

FIG. 9 are additional graphs reporting measurements performed using the sensor system of the present invention.

### PREFERRED EMBODIMENT OF THE INVENTION

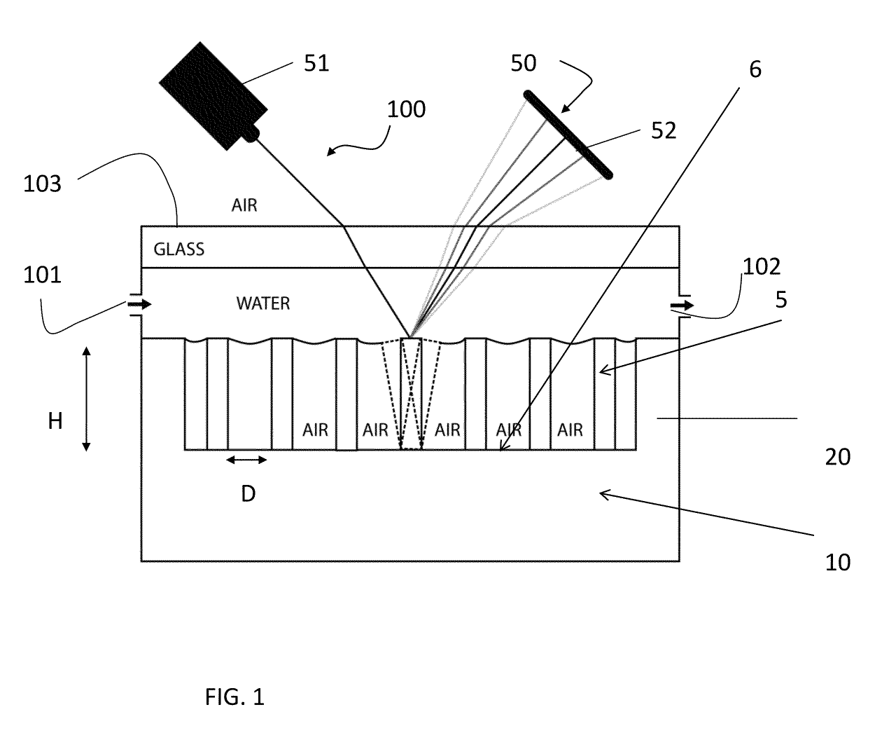

With initial reference to FIG. 1, which 100 a sensor system according to the invention is globally indicated.

The sensor system 100 includes a sensor 10 having a plurality of pillars 5. The pillars 5 can be distributed randomly or according to a given regular pattern.

Pillars 5 vertically protrudes from a substrate 6. Pillars 5 and substrate 6 might be realized by the same material or by different materials. Preferably, pillars 5 are realized in silicon or crystalline silicon, silicon carbide, silicon nitride, carbon compound (including polymers), III-V compounds or any or a combination of the materials which are easily fabricated with standard lithography and etching processes and offer a high young modulus or silicon on insulator.

Each pillar 5 includes a base 5b attached to the substrate 6, which can also be realized en bloc with the substrate itself, and a second base 5a which is free and substantially parallel to the substrate itself. The second base 5a has preferably an area of 0.2 μm² and 50 μm². In addition each pillar comprises a lateral surface 5c connecting the free base with the substrate 6.

The height H of each pillar 5 is preferably comprised between 5 μm and 50 μm, while the distance D between any two nearest neighbors (shortly n.n.) of the plurality of pillars is such that that H/D<1, preferably 2<H/D<5, where H is the height of any of the n.n. pillars and D their distance. The height H of pillars 5 can be the same or it can vary among the pillars in the plurality, as long as the above mentioned equation is satisfied. In addition, the distance D between n.n. pillars can be random within a given range, such range being selected so that the above equation is always satisfied, or can be always the same as in a regular lattice.

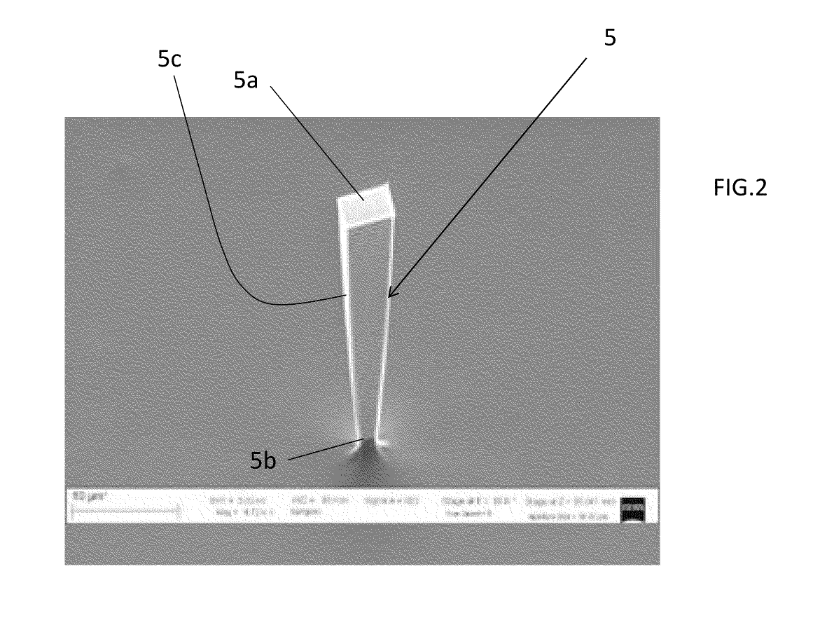

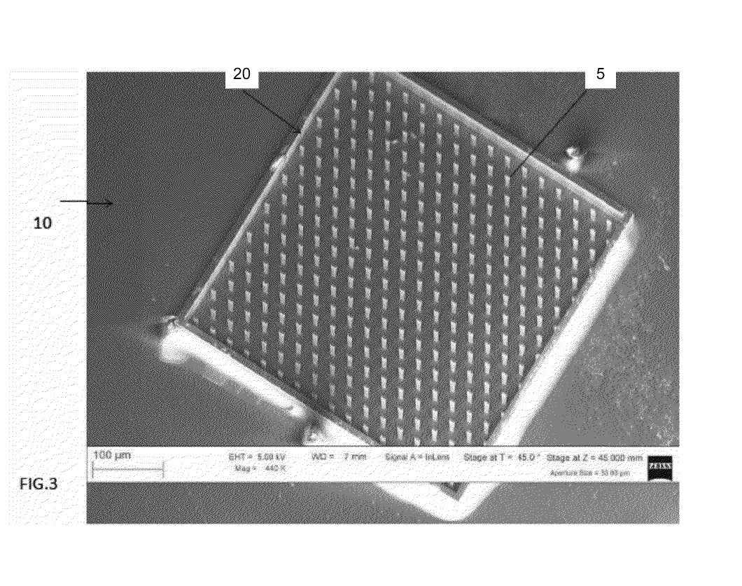

An hexagonal array of pillars is shown as an example in FIG. 3, while a single pillar 5 of the plurality in shown in the SEM image of FIG. 2.

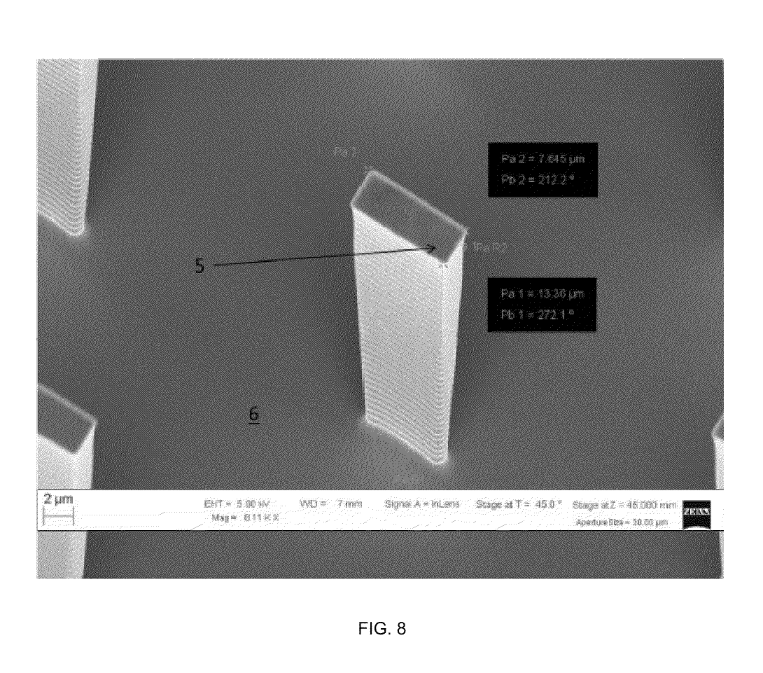

Preferably, pillars 5 have a rectangular cross section and a frusto-conical shape, i.e. the value of the area of the cross section is reduced to the minimum at the base 5b attached to the substrate 6 and it reaches the maximum value in correspondence to its free end 5a. The frusto-conical shape can be easily seen in the SEM photograph of FIG. 8. The slight undercut allows to reduce the sensor mass without reducing the active area of the sensor itself, the active area being the top free end surface 5a as detailed below. In addition, the small base 5b increases the pillar oscillations. The stress is confined to the pillar base.

The free end 5a of the pillar 5 is functionalized. In particular, preferably the pillar's free end 5a is coated with a metallic layer (not visible in the photographs), for example a layer of Chromium Gold. Silane functionalization can be alternatively applied to bare silicon surface 5a. Analogous functionalization can be applied to different materials than silicon. Alternatively, other functionalizations can be used, for example the free end surface can be functionalized using organic molecules having a functional group that in turn can be linked to the compound of interest.

The thickness of the metallic layer is comprised between 10 nm to 50 nm. In addition the surface 5a is hydrophilic. Preferably this is achieved via the same functionalization treatment.

The lateral surface 5c of the pillars is hydrophobic. This is preferably obtained via a coating of a layer of hydrophobic material (also this layer is not shown).

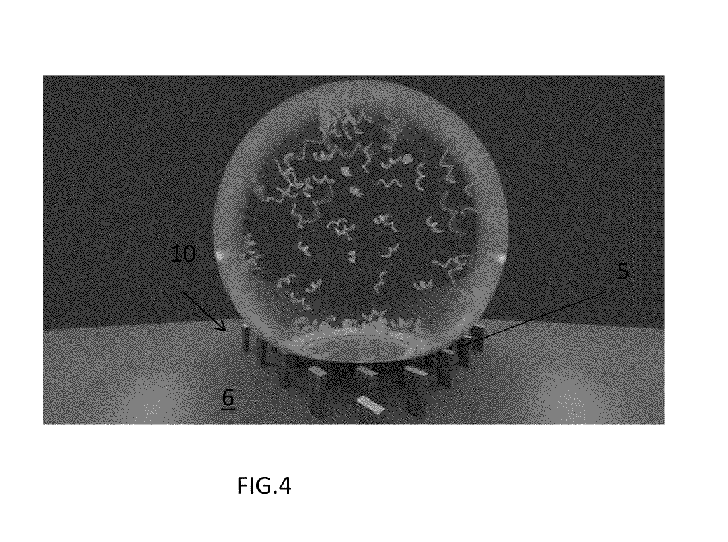

The fact that the ratio between height and distance of n.n. pillars and their height is smaller than 1, and that the lateral surface of the pillar is hydrophobic give to the sensor 10 a super-hydrophobic behavior when liquid is coming into contact with the pillars 5. This effect is schematically depicted on FIG. 4, where a single droplet of liquid on top of the pillars is shown: due to the super hydrophobic effect, the substrate 6 and the lateral surface 5c of pillars 5 do not get wet, only the functionalized free bases 5a are in contact with the liquid.

Pillars are surrounded by a wall 20, the distance between the wall and the pillars is so that the nearest neighbor pillars to the wall have a height which is larger than their distance.

The pillars are obtained using the following method. Preferably, they are obtained using two different steps starting from a substrate 6: the first one is the definition of their cross section via lithography and then an etching phase to etch the substrate till the desired depth.

Example of Pillar Fabrication

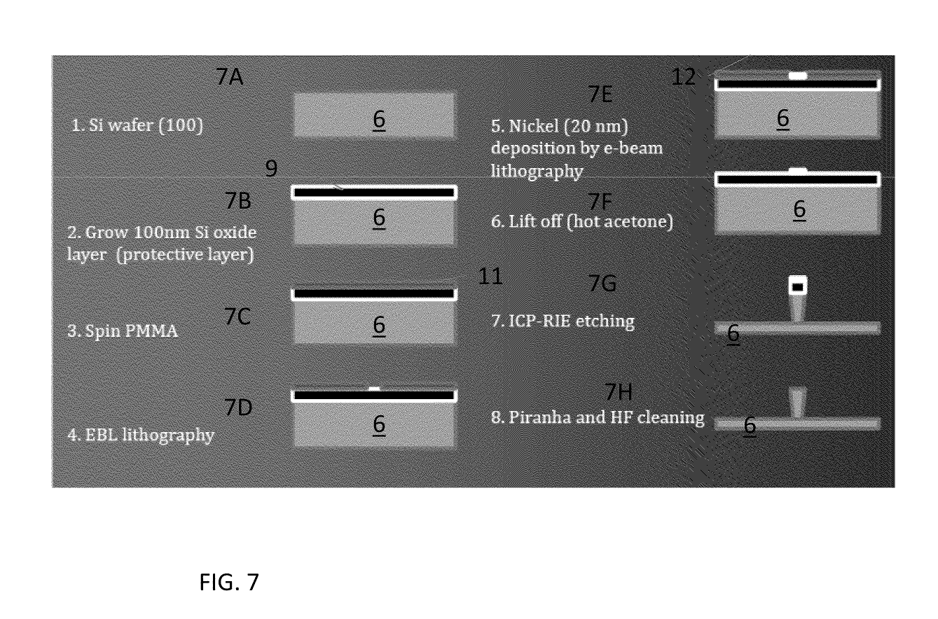

The fabrication of the pillars 5 of sensor 10 is described with reference to FIG. 7.

Prior to the lithography and etching, the substrate 6 undergoes additional cleaning and surface preparation steps.

The starting material is a (1 0 0) oriented, single side polished, P-type silicon wafer which represents the substrate 6 and also the material in which the pillars 5 are realized (step 7A). After piranha (H₂O₂ (35%):H₂SO₄=1:3 at 90° C.) and HF cleaning, a 100 nm silicon dioxide layer 9 is grown by Plasma Enhanced Chemical Vapor Deposition (step 7B). The silicon oxide layer 9 has the function of protecting from contaminations and defect produced during the fabrication process, the portion of the substrate which will be the top area (i.e. the free end 5a) of the pillar 5.

The sample is spin coated with 500 nm of poly-methylmethacrate (PMMA) 950 K resist (4000 rpm) 11 (step 7C) and baked for 10 min at 180° C.

The pillar in-plane geometry and the overall patterning are defined by e-beam lithography (Zeiss Leo 30 keV), step 7D. A rectangular cross section has been chosen: the spectrum of mechanical response of such a configuration shows one well defined peak.

After PMMA developing in a conventional 1:3 MIBK/IPA developer for one minute, a 20 nm nickel layer 12 is evaporated by means of e-beam and the Ni mask for the subsequent dry etching is obtained through a lift off process by removing the resist in hot acetone (step 7E). Before the etching, oxygen plasma is performed in order to remove the residual resist and argon plasma is used to define better the metal mask. A Bosch™-like process to obtain a deep etching for both silicon and silicon oxide with an Inductively Coupled Plasma reactor (ICP, STS-Surface Technology) has been developed. For passivation, plasma of mixture of C4F8 and Ar (100 and 20 sccm) at a pressure of 7 mTorr and with 600 W of RF power applied to the coil is used.

For etching a plasma of mixture of SF6 and Ar (110 and 20 sccm) at a pressure of 8 mTorr and with 600 W of RF power applied to the coil and 50 W to the platen is used. Many cycles are executed to remove silicon to create the vertical resonator. The duration of the process settles the height of the pillar. Typically, almost 15 μm which correspond to 48 cycles are removed.

It is preferred to avoid a strictly vertical profile. So the etching process has a small undercut (≈4°, see FIG. 8 already mentioned). Normally this is an undesired effect but in this case it results in a inverted tapered profile that has several advantages: the sensor mass is reduced (about 50%) without changing the sensitive area (which corresponds to the area of surface 5a); the structure in insensitive to small misalignment during the top gold evaporation because of the intrinsic shadowing effect; the oscillation amplitude is increased, due to the thin pillar base; the stress induced by the oscillation is confined on the pillar base, which is less affected by the thermal drift induced by the laser which is used for monitoring the motion of the pillar (step 7G).

The metal mask and protective silicon oxide are removed providing a clean and flat silicon surface for the next functionalization process. First, the metal mask is dissolved by a 15 min dipping in piranha solution, and then the oxide is dissolved in hydrofluoric acid (step 7H). The final step is to re-oxidize the devices which are put for 1 hour and half in furnace at 1100° C. in gentle flux of water vapor.

The result is shown in FIGS. 2 and 8. The etching time was chosen to achieve pillars of height 5 μm. Typical dimensions of the cross section are 3 μm×5 μm or 3 μm×8 μm at the free end 5a. The lateral wall are not vertical respect to the substrate but are tilted. At the base the cross section is reduced to 0.8 μm×2 μm or 0.8 μm×6 μm.

A sensor device 100 with hexagonal lattice pattern has been fabricated. The lattice is made by 19 rows with 16 pillars (see FIG. 3). The distance between to neighbor pillar is 1 μm and the height is 5 μm. The matrix is enclosed in a square corral 20 (the wall) that avoids water entering laterally at the bottom of the structure.

The end surface 5a of each pillar is functionalized through incubation in 1 micromolar solution of alcanethiolated molecules for at least one hour. This is preferably done after the following step of hydrophobicization of the lateral surfaces 5c.

The third, fundamental, fabrication step consists in the hydrophobication of the structure. Indeed, only when the material itself shows contact angle in excess of 90° for a flat surface, the microstructuring gives super hydrophobicity. The lateral surfaces 5c of the pillars 5 is made hydrophobic by depositing a layer of Teflon by plasma assisted polymerization of C₄F₈ gas.

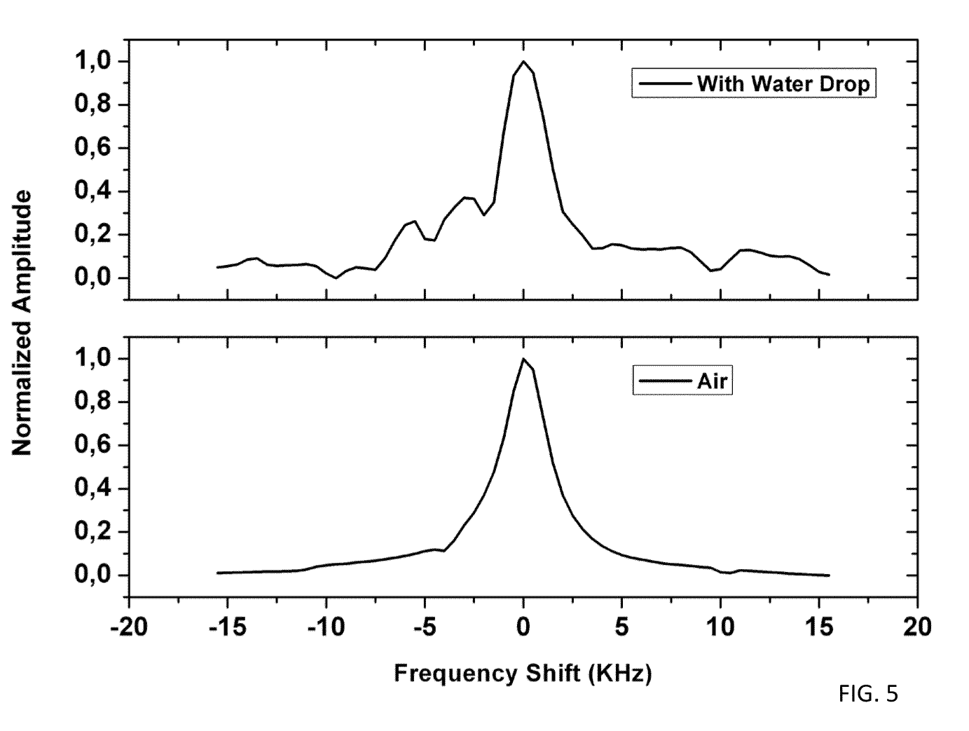

It worth to note that atmospheric pressure pillars realized according to the above still have a relatively high Q-factor, around 1000. This is shown in FIG. 5 where Delta f is about 5 kHz and the pillar resonance is at about 5 Mhz.

With now reference back to FIG. 1, the sensor system 100, in addition to sensor 10, includes a detection device 50 used to detect the oscillations of pillars 5. On the pillars 5, and in particular on their free end surface 5a, a laser beam impinges.

The sensor formed by pillars 5 is one of the element of a microfluidic chamber (see FIG. 1) where wall 20 defines the lateral delimitation of the chamber and substrate 6 the bottom. A fluid is introduced on top of the pillars via an inlet 101 in order to detect the target compound(s) and exits the camber via outlet 102. The fluid is kept flowing by a pumping system (not shown). The chamber is closed by a top wall 103 realized at least partially by a material transparent to the laser light which will be reflected on top of one of the pillars 5, such as glass. The top wall can be also be realized completely by glass.

Between the introduced fluid and the pillars, i.e. in contact with the lateral walls 5a of the pillars 5 air or any other suitable gas is present. The fluid is in contact substantially only with the top surfaces 5a of pillars 5.

The oscillation of the pillars 5, as said, changes depending on the mass of a molecule that attaches on the functionalized surface 5a.

As a detection device 50l any known method can be used. Preferably, as said, a laser 51 emits a laser beam toward the pillars' free surface 5a and a detector, such as a photodiode 52, collects the reflected light which is then analyzed (see FIG. 1).

Optical Set/Up

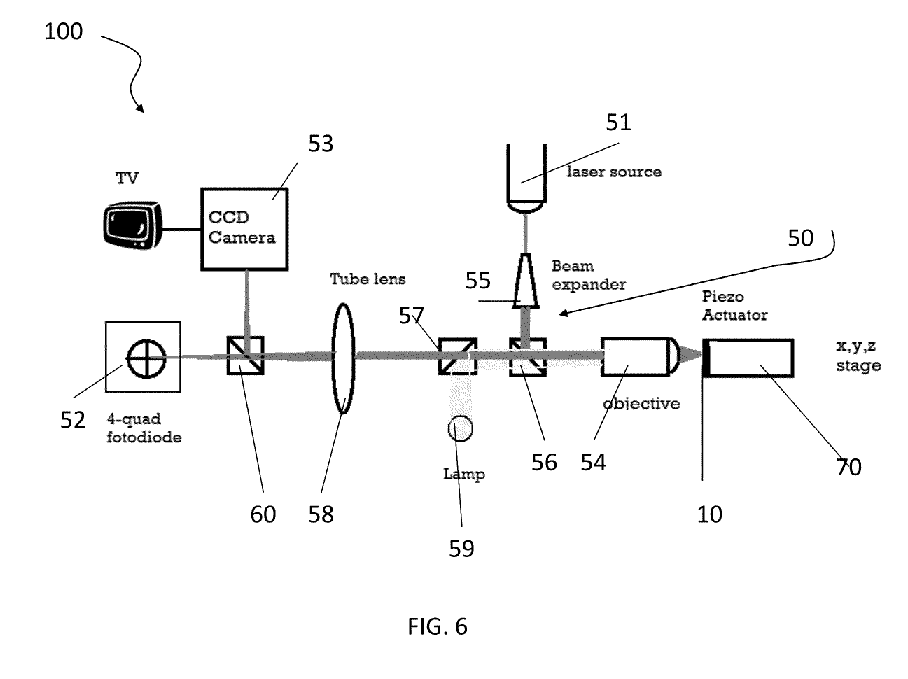

The optical setup is depicted in FIG. 6. It is build using the cage system (from Thorlabs™) that consists in a rigid armature of four steel rods, where the optical components are mounted along a common optical axis. The distance between two near rods is 30 mm. The setup serves the purpose to focus a laser beam 51 in a spot of few microns, to focus on a photodetector 52 the light reflected from a pillar 5 (see FIG. 1) and to visualize by means of a CCD camera 53 the laser spot and the device. The source is a DPSS green laser (532 nm) that can be modulated from 0 to 100 mW. A relatively high power is needed because of the several reflections along the optical path that reduce the actual power reflected by the pillar 5 on the photodetector 52. Almost 1/10th of the incident power reaches the pillar surface 5a. A long working distance microscope objective 54 (LMPLFLN 20X Olympus) with 0.4 numerical aperture and 12 mm working distance focuses the laser to a spot of few microns. The diameter of the entrance pupil of the objective is around 7 mm and the beam radius of the laser must be expended in order to illuminate all the optics of the objective. For this a 10× beam expander 55 is mounted between the laser 51 and the objective 54. A cubic beam splitter 56 divides the incident and the reflective light. A tube lens 58, (focal lens 200 mm) is used to correct the infinity focus of the objective. A second beamsplitter 57 serves the purpose to add a white light in optical path for the illumination. The source 59 is a common fiber optic illuminator. A mirror 60, after the tube lens, can direct the light either to the photodetector 52 or to the CCD camera 53 (GANZTM ZCF11C4 or THEIMAGINGSOURCETM DBK41BU02). Alternatively, with a further beam splitter (also noted with 60) it is possible to achieve the imaging and the detection at the same time with the drawback to halve the signal on the photodiode. Before the CCD camera a long pass filter (610 nm) stops the laser light allowing only the imaging light to reach the detector otherwise the laser intensity would saturate the sensor of the camera. The portion of the incident light that pass through the beam splitter orthogonally respect to the objective is monitored by a power meter sensor.

The optical system is fixed and the scanning over the sample is realized by moving the entire chamber by means of a xy micrometric translation stage and on a lab jack. A second xyz stage controls the position of the photodetector 52. Moreover a high precision rotation stage can turn the sensor around the optical axis of the system. The sample holder is designed to be fast placed by means of a dovetail sliding interlocking.

Preferably, the sensor 10 is put into oscillation to increase the accuracy of the measurements. More preferably, the oscillations are generated by a piezoelectric.

The chips including the sensor 10 are mounted on a chips support made of PEEK with four chip-slots equipped with four 3×5×1 mm piezoelectric crystal (lead zirconate titane) which are used as actuators 70. Their capacity ranges from 0.5 nF to 1.2 nF. The samples are directly glued to the crystals by means of bi adhesive tape.

It has been tested that the direction of vibration of the piezo 70 has a small influence on the motion (oscillations) of a pillar 5.

The photodector 52 has a fast four quadrant photodiode (Hamamatsu S7379-01, cut off frequency≈80 MHz) and a dedicated homemade electronics. The four signals of the four quadrants are amplified and mixed generating two outputs: the x and y positions of the spot respect to the center of the photodiode. These values are proportional to the displacement of the illuminated pillar. By monitoring the two signals with a multi-channel oscilloscope the photodiode is aligned with the laser beam.

A network analyzer (3577A Hewlett-Packard), not shown, generates a sweeping signal which excites the piezo that makes the pillars to oscillate. Depending on the orientation of the pillar, the vertical or the horizontal signal is acquired by the analyzer which filters the component of the signal at the actuation frequency and provides the amplitude and the phase difference. The instruments allow collecting 401 points and the typical frequency span is 10 KHz. The duration of the sweep is 60 second.

A periodic collecting of the spectra for a specific duration is preferably made. Typically, the rate is every 2 minutes for 20 minutes. This time is enough to obtain a stable value. By fitting the data with a lorentzian function the center xc and the width w give the value of the resonance frequency (xc) and of the Q-factor (xc/w).

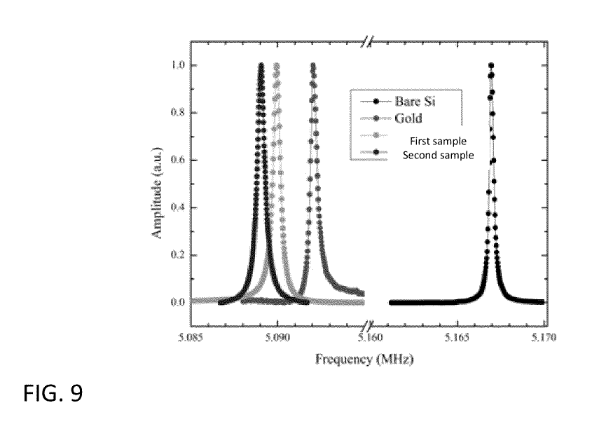

As shown in FIG. 5, using the above sensor system and detecting the oscillation of one of the pillars 5, there is substantially no shift between the resonance of the pillar in water and in vacuum, as demonstrated by comparing the two curves. Some measurements performed with such a sensor system are shown in FIG. 9. The different curves shows the different frequencies at which resonance is present. The right-most curve represent the peak of the pillar 5 (“bare silicon”) before the functionalization of the top surface 5a. The second right-most curve represents the peak of the same pillar after functionalization (i.e. after the gold deposition on surface 5a). The frequency shift between the two curves represents an added mass of: 2242 femtograms.

The second curve from left represents the measurements of the same pillar after a monolayer of thiolated ssDNA 40 base-pairs long has been formed through 1 h incubation in 1 micromolar solution of the latter: the frequency shift between the two curves (the “first sample” and the “gold” curves) represents an added mass of 600 fg.

The first curve from left represents the measurements of the same pillar where the ssDNA monolayer has been exposed for one hour to a 1 micromolar solution of the complementary DNA sequence: the frequency shift between the two curves (the “second sample” and the “first sample” curves) represents an added mass of 300 fg and indicates that roughly 50% of the DNA is hybridized.

1. A sensor system to detect the mass of a compound in a liquid solution, said system comprising:
a sensor including a plurality of pillars extending from a substrate and having a given height,
said pillars having a free end opposite to the substrate, and including a lateral surface connecting said free end to said substrate,
said free end defining a surface and said surface being functionalized in order to bind with said compound to be detected, and said lateral surface being hydrophobic,
wherein the distance between any two nearest neighbors pillars of the plurality satisfies the following equation
$$\frac{height of any of the two n . n . pillars}{maximum distance between the two n . n . pillars} > 1$$
a detection device to detect the oscillations of said pillars.

2. The sensor system according to claim 1, wherein the equation to be satisfied is
$$2 < \frac{height of any of the two n . n . pillars}{maximum distance between the two n . n . pillars} > 5$$

3. The sensor system of claim 1, wherein the distance between two nearest neighbor pillars is comprised between 2 μm and 50 μm.

4. The sensor system of claim 1, wherein the height of a pillar of the plurality is comprised between 5 μm and 50 μm.

5. The sensor system according to claim 1, wherein said plurality of pillars are surrounded by a wall protruding from said substrate.

6. The sensor system according to claim 5, wherein the height of said wall is substantially the same as the height of any of said pillars.

7. The sensor system according to claim 5, wherein the maximum distance between the wall and each of its nearest neighbor pillars satisfies the following equation:
$$\frac{height of any of the nearest neighbor pillar and the wall}{maximum distance between the wall and the n . n . pillars} > 1$$

8. The sensor system according to claim 5, wherein the maximum distance between the wall and each of its nearest neighbor pillars satisfies the following equation:
$$2 < \frac{height of any of the nearest neighbor pillar and the wall}{maximum distance between the wall and the n . n . pillars} > 5.$$

9. The sensor system according to claim 1, wherein the pillar is frusto-conical, having a cross sectional area which increases starting from the substrate towards the free end surface.

10. The sensor system according to claim 9, wherein the angle formed by the lateral surface and the substrate is comprised between 3° and 6°.

11. The sensor system according to claim 1, wherein said free end surface includes a layer of metallic material.

12. The sensor system according to claim 1, wherein said sensor is super hydrophobic.

13. The sensor system according to claim 12, wherein said lateral surface is coated with a water-repellent material.

14. The sensor system according to claim 1, wherein said free end surface is hydrophilic.

15. The sensor system according to claim 1, wherein said pillar and/or said substrate includes silicon.

16. The sensor system according to claim 1, including a microfluidic chamber wherein said sensor is the bottom element, said microfluidic chamber comprising:
an inlet and an outlet port for the flow of the fluid including the target compound,
an upper wall made at least partially of an optically transparent material.

17. A sensor system according to claim 16, wherein said microfluidic chamber has an overall liquid volume comprised between 0.01 nL and 10 nL.

18. A sensor system according to claim 16 or claim 17, wherein said upper wall of said microfluidic chamber has a water repellent functionalization to avoid specific wavelength absorption.

19. The sensor system according to claim 1, wherein said detection device includes a laser to impinge a laser beam onto a free end surface of one of the pillars of said plurality and a photodetector to detect the reflected light.

20. The sensor system according to claim 19, wherein said laser beam crosses said top wall of said microfluidic chamber.

21. The sensor system according to claim 1, including an actuator to put said sensor into oscillations.

22. The sensor system according to claim 21, wherein said actuator is a piezoelectric device.

23. The sensor system according to claim 1, wherein said compound is a molecule.

24. The sensor system according to claim 23, wherein said molecule is an analyte.
